(19) United States
(12) Patent Application Publication
(10) Pub. No.: US 2024/0285823 A1
(43) Pub. Date: Aug. 29, 2024

(54) STERILIZATION SYSTEM AND METHOD USING IONIZED HYDROGEN PEROXIDE

(71) Applicant: TOMI ENVIORNMENTAL SOLUTIONS, INC., FREDERICK, MD (US)

(72) Inventors: Halden Stuart Shane, Beverly Hills, CA (US); Elissa J. Shane, Frederick, MD (US); Johnny Sullivan Cato, Lynwood, CA (US)

(21) Appl. No.: 18/586,983

(22) Filed: Feb. 26, 2024

Related U.S. Application Data
(60) Provisional application No. 63/486,744, filed on Feb. 24, 2023.

(51) Int. Cl. A61L 2/18; A61L 2/14; A61L 2/24; A61L 2/26

(52) U.S. Cl. CPC A61L 2/18; A61L 2/14; A61L 2/24; A61L 2/26

(57) ABSTRACT

A method for decontaminating a closed space, comprising providing a reservoir of a cleaning fluid; generating a very dry mist comprising cleaning fluid comprising aerosol droplets, wherein the mist is subjected to a nonthermal plasma actuator to form plasma activated ionic particles carried by the aerosol droplets of the mist, wherein the plasma activated ionic particles are hydroxyl ions; wherein a flow meter monitors a flow of the mist through a self-cleaning nozzle into the closed space, and wherein the flow rate of the mist through the self-cleaning nozzle is not greater than 2 ml/min; wherein air pressure within the closed space is maintained as neutral; wherein there is no cavity permitted within the self-cleaning nozzle other than that through which the very dry mist flows, wherein the generated very dry mist is applied to decontaminate the closed space.

This application claims priority to U.S. Provisional Application No. 63/486,744, filed Feb. 24, 2023. The entirety of the aforementioned applications is incorporated herein by reference.

### FIELD

The present application relates generally to a method for sterilizing spaces related to devices, systems, and methods of manufacturing cell products for biomedical applications using automated systems.

### BACKGROUND

Microbiological species are widely distributed in our environment. Most microbiological species are of little concern, because they do not damage other living organisms. However, other microbiological species may infect man or animals and cause them harm. The removing or rendering ineffective of injurious microbiological organisms has long been of interest. Drugs and medical system described hereins are sterilized and packaged in sterile containers. Medical environments such as operating rooms, wards, and examination rooms are decontaminated by various cleaning procedures so that injurious microbiological organisms cannot spread from one patient to another.

Many available technologies for controlling microbiological organisms are of limited value in the public health circumstances of biological warfare and bioterrorism. Furthermore, current technologies addressing these instances are limited in their effectiveness in tightly enclosed environments.

One of the challenges of controlling microbiological organisms relates to sterilizing closed spaces. In such environments, it is not uncommon for a mist of decontaminants to travel on compressed air as far as several feet under normal settings. In a closed space, excessive reach of the sprayed mist results in several undesired outcomes. One such drawback is saturating surfaces in the vicinity of the mist applicators, or surfaces opposite to the applicators, for example. Moreover, improper mist regulation may result in wetter, denser fog, thereby affecting visibility and breathability in a closed space. Accordingly, the released mist undesirably increases moisture accumulation and condensation, the redressing of which requires increased aeration times.

Cellular therapies based on hematopoietic stem cells (HSCs), chimeric antigen receptor (CAR) T cells, NK cells, tumor infiltrating lymphocytes (TILs), T-cell receptors (TCRs), regulatory T cells (T regs), gamma delta (γδ) T cells, and others rely on manufacturing of cell products. Manufacturing of such cell products typically involves multiple cell processing steps. Conventional solutions for manufacture of cell products rely on cumbersome manual operations performed in expensive biosafety system described hereins and/or clean rooms. Skilled laboratory technicians, adequate sterile enclosures such as cleanroom facilities, and associated protocols and procedures for regulated (GMP) manufacturing are expensive. Many current manufacturing processes employ numerous manual reagent preparation and instrument manipulation steps during a manufacturing protocol, and the processes may require several days or even weeks. Even platforms described as automated cell processing in a closed system generally rely on pre-configured instrumentation and tubing sets that limit operational flexibility and do not reliably prevent process failure due to accidental operator/human error. Contamination of such spaces is fatal to the process.

### SUMMARY

An aspect of the application is a method for decontaminating a closed space, comprising the steps of: entering input parameters of the closed space into a processing unit, wherein the processing unit is programmed to determine fluid properties of a cleaning fluid in a decontamination device based on the input parameters of the closed space, activating a decontamination cycle of the decontamination device, wherein the decontamination cycle comprises the steps of: providing a reservoir of the cleaning fluid; setting the determined fluid properties of the cleaning fluid; generating a very dry mist comprising cleaning fluid at substantially one atmosphere ambient pressure comprising aerosol droplets, wherein the cleaning fluid comprises a source of an active species for sterilization, wherein the active species is hydroxyl ions and wherein the source is hydrogen peroxide, and the cleaning fluid is silver-free chlorine-free and peracetic-acid free; wherein the mist is subjected to a nonthermal plasma actuator to form plasma activated ionic particles carried by the aerosol droplets of the mist, wherein the plasma activated ionic particles are hydroxyl ions; wherein a flow meter monitors a flow of the mist through a self-cleaning nozzle into the closed space, and wherein the flow rate of the mist through the self-cleaning nozzle is not greater than 2 ml/min; wherein a pump controls flow rates and pressure through the self-cleaning nozzle by a pump, wherein air pressure within the closed space is maintained as neutral; wherein the self-cleaning nozzle receives the flow of the mist through tubing of not greater than ⅛″ and there is no cavity permitted within the self-cleaning nozzle, wherein the generated very dry mist is applied to decontaminate the closed space.

An aspect of the application is system for decontaminating a closed space, comprising a decontamination device and a computer processor, wherein the computer processor is in networked communication with the decontamination device, wherein input parameters of the closed space are entered into the computer processor, wherein the computer processor is programmed to determine fluid properties of a cleaning fluid in the decontamination device based on the input parameters of the closed space, wherein the computer processor is further programmed to activate a decontamination cycle of the decontamination device, the decontamination cycle comprising the steps of: providing a reservoir of the cleaning fluid; setting the determined fluid properties of the cleaning fluid; generating a very dry mist comprising cleaning fluid at substantially one atmosphere ambient pressure comprising aerosol droplets, wherein the cleaning fluid comprises a source of an active species for sterilization, wherein the active species is hydroxyl ions and wherein the source is hydrogen peroxide, and the cleaning fluid is silver-free chlorine-free and peracetic-acid free; wherein the mist is subjected to a nonthermal plasma actuator to form plasma activated ionic particles carried by the aerosol droplets of the mist, wherein the plasma activated ionic particles are hydroxyl ions; wherein a flow meter monitors a flow of the mist through a self-cleaning nozzle into the closed space, and wherein the flow rate of the mist through the self-cleaning nozzle is not greater than 2 ml/min; wherein a pump controls flow rates and pressure through the self-cleaning nozzle by a pump, wherein air pressure within the closed space is maintained as neutral; wherein the self-cleaning nozzle receives the flow of the mist through tubing of not greater than ⅛″ and there is no cavity permitted within the self-cleaning nozzle, wherein the generated very dry mist is applied to decontaminate the closed space.

These and other aspects and embodiments of the present application will become better understood with reference to the following detailed description when considered in association with the accompanying drawings and claims.

### BRIEF DESCRIPTION OF THE DRAWINGS

FIG. 1 represents an architecture of the sterilization system components and their relationships.

FIG. 2 shows a sterilization systems flow chart.

FIG. 3A shows an applicator design for decontamination of a closed space from an external viewpoint. FIG. 3B shows an applicator design for the same applicator showing the internal arrangements.

FIG. 4A shows a fitting. FIG. 4B shows the process of potting the fitting to reduce internal diameter.

FIG. 5A shows front view of self-cleaning nozzle. FIG. 5B shows side view of self-cleaning nozzle.

FIG. 6 shows the operation of a self-cleaning nozzle.

Throughout the drawings, the same reference numerals and characters, unless otherwise stated are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and appended claims.

### DETAILED DESCRIPTION

Reference will be made in detail to certain aspects and exemplary embodiments of the application, illustrating examples in the accompanying structures and figures. The aspects of the application are described in conjunction with the exemplary embodiments, including methods, materials and examples, such description is non-limiting and the scope of the application is intended to encompass all equivalents, alternatives, and modifications, either generally known, or incorporated here. With respect to the teachings in the present application, any issued patent, pending patent application or patent application publication described in this application is expressly incorporated by reference herein.

Unless defined otherwise, all technical and scientific terms used herein have the same meanings as commonly understood by one of skill in the art to which the disclosed method and compositions belong. It must be noted that as used herein and in the appended claims, the singular forms “a,” “an,” and “the” include plural reference unless the context clearly dictates otherwise. Thus, for example, reference to “a peptide” includes “one or more” peptides or a “plurality” of such peptides.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

### Definitions

As used herein, the term “sterilizing” means acting to neutralize or remove pathogens from an area or article. As used herein, the term “pathogen” includes, but is not limited to, a bacterium, viruses, yeast, protozoan, or other pathogenic microorganisms. The term “pathogen” also encompasses targeted bioterror agents.

As used herein, the term “bacteria” shall mean members of a large group of unicellular microorganisms that have cell walls but lack organelles and an organized nucleus. Synonyms for bacteria may include the terms “microorganisms”, “microbes”, “germs”, “bacilli”, and “prokaryotes.” Exemplary bacteria include, but are not limited to Mycobacterium species, including M. tuberculosis; Staphylococcus species, including S. epidermidis, S. aureus, and methicillin-resistant S. aureus; Streptococcus species, including S. pneumoniae, S. pyogenes, S. mutans, S. agalactiae, S. equi, S. canis, S. bovis, S. cquinus, S. anginosus, S. sanguis, S. salivarius, S. mitis; other pathogenic Streptococcal species, including Enterococcus species, such as E. faecalis and E. faecium; Haemophilus influenzae, Pseudomonas species, including P. aeruginosa, P. pseudomallei, and P. mallei; Salmonella species, including S. enterocolitis, S. typhimurium, S. enteritidis, S. bongori, and S. choleraesuis; Shigella species, including S. flexneri, S. sonnei, S. dysenteriae, and S. boydii; Brucella species, including B. melitensis, B. suis, B. abortus, and B. pertussis; Neisseria species, including N. meningitidis and N. gonorrhoeae; Escherichia coli, including enterotoxigenic E. coli (ETEC); Vibrio cholerae, Helicobacter pylori, Geobacillus stearothermophilus, Chlamydia trachomatis, Clostridium difficile, Cryptococcus neoformans, Moraxella species, including M. catarrhalis, Campylobacter species, including C. jejuni; Corynebacterium species, including C. diphtheriac, C. ulcerans, C. pseudotuberculosis, C. pscudodiphtheriticum, C. urcalyticum, C. hemolyticum, C. equi; Listeria monocytogenes, Nocardia asteroides, Bacteroides species, Actinomycetes species, Treponema pallidum, Leptospirosa species, Klebsiella pneumoniae; Proteus sp., including Proteus vulgaris; Serratia species, Acinetobacter, Yersinia species, including Y. estis and Y. pseudotuberculosis; Francisella tularensis, Enterobacter species, Bacteroides species, Legionella species, Borrelia burgdorferi, and the like. As used herein, the term “targeted bioterror agents” includes, but is not limited to, anthrax (Bacillus antracis), plague (Yersinia pestis), and tularemia (Franciscella tularensis).

As used herein, the term “virus” can include, but is not limited to, influenza viruses, herpesviruses, polioviruses, noroviruses, and retroviruses. Examples of viruses include, but are not limited to, human immunodeficiency virus type 1 and type 2 (HIV-1 and HIV-2), human T-cell lymphotropic virus type I and type II (HTLV-I and HTLV-II), hepatitis A virus, hepatitis B virus (HBV), hepatitis C virus (HCV), hepatitis delta virus (HDV), hepatitis E virus (HEV), hepatitis G virus (HGV), parvovirus B19 virus, hepatitis A virus, hepatitis G virus, hepatitis E virus, transfusion transmitted virus (TTV), Epstein-Barr virus, human cytomegalovirus type 1 (HCMV-1), human herpesvirus type 6 (HHV-6), human herpesvirus type 7 (HHV-7), human herpesvirus type 8 (HHV-8), influenza type A viruses, including subtypes H1N1 and H5N1, human metapneumovirus, severe acute respiratory syndrome (SARS) coronavirus, hantavirus, and RNA viruses from Arenaviridac (e.g., Lassa fever virus (LFV)), Pneumoviridae (e.g., human metapneumovirus), Filoviridae (e.g., Ebola virus (EBOV), Marburg virus (MBGV) and Zika virus); Bunyaviridae (e.g., Rift Valley fever virus (RVFV), Crimcan-Congo hemorrhagic fever virus (CCHFV), and hantavirus); Flaviviridae (West Nile virus (WNV), Dengue fever virus (DENV), yellow fever virus (YFV), GB virus C (GBV-C; formerly known as hepatitis G virus (HGV)); Rotaviridae (e.g., rotavirus), and combinations thereof. In one embodiment, the subject is infected with HIV-1 or HIV-2. As used herein, the term “fungi” shall mean any member of the group of saprophytic and parasitic spore-producing eukaryotic typically filamentous organisms formerly classified as plants that lack chlorophyll and include molds, rusts, mildews, smuts, mushrooms, and yeasts. Exemplary fungi include, but are not limited to, Aspergillus species, Dermatophytes, Blastomyces derinatitidis, Candida species, including C. albicans and C. krusei; Malassezia furfur, Exophiala werneckii, Piedraia hortai, Trichosporon beigelii, Pseudallescheria boydii, Madurella grisca, Histoplasma capsulatum, Sporothrix schenckii, Histoplasma capsulatum, Tinea species, including T. versicolor, T. pedis T. unguium, T. cruris, T. capitus, T. corporis, T. barbac; Trichophyton species, including T. rubrum, T. interdigitale, T. tonsurans, T. violaceum, T. yaoundei, T. schoenleinii, T. megninii, T. soudanense, T. equinum, T. erinacei, and T. verrucosum; Mycoplasma genitalia; Microsporum species, including M. audouini, M. ferrugineum, M. canis, M. nanum, M. distortum, M. gypseum, M. fulvum, and the like.

As used herein, the term “protozoan” shall mean any member of a diverse group of eukaryotes that are primarily unicellular, existing singly or aggregating into colonies, are usually nonphotosynthetic, and are often classified further into phyla according to their capacity for and means of motility, as by pseudopods, flagella, or cilia. Exemplary protozoans include, but are not limited to Plasmodium species, including P. falciparum, P. vivax, P. ovale, and P. malariae; Leishmania species, including L. major, L. tropica, L. donovani, L. infantum, L. chagasi, L. mexicana, L. panamensis, L. braziliensis and L. guyanensi; Cryptosporidium, Isospora belli, Toxoplasma gondii, Trichomonas vaginalis, and Cyclospora species.

As used herein, the term “cell processing device” means any man-made structure that is a closed space and may be susceptible to contamination with pathogens. The term “cell processing device” is not limited to man-made structures, even though embodiments illustrated herein may be preferably directed to sterilization of such structures.

As used herein, the term “sensor” can refer to any type of sensor suitable for detecting contamination on an apparatus, a surface, or in a substantially closed space. Examples of sensors include, but are not limited to, photosensors, voltaic sensors, weight sensors, moisture sensors, pressure sensors, or any type of biosensor.

As used herein, the term “shearing” refers to the process of using force to fragment liquid particles into discrete groups that move and flow as energized independent sub-groups of sheared particles until the groups of particles transition in fluid phase into a mist. As used herein, the term “mist” means a cloud of aerosol droplets. As used herein, the term “aerosol” is a colloid of fine liquid droplets of about 1 to about 20 micrometers in diameter.

As used herein, the term “cleaning fluid” or “sterilization fluid” refers to the source of an active species used to decontaminate an article or cell processing device. The preferred active species is hydroxyl ions, and the preferred source is hydrogen peroxide. The source may instead be a more-complex species that produces hydroxyl ions upon reaction or decomposition. Examples of such more-complex species include peracetic acid (CH₂COO—OH+H₂O), sodium percarbonate (2Na₂CO₃+3H₂O₂), and gluteraldehyde (CH₈O₂). The cleaning fluid may further include promoting species that aid the active species in accomplishing its attack upon the biological microorganisms. Examples of such promoting species include ethylenediaminetetraacetate, isopropyl alcohol, enzymes, fatty acids, and acids. The cleaning fluid is of any operable type. The cleaning fluid must contain an activatable species. A preferred cleaning fluid comprises a source of hydroxyl ions (OH⁻) for subsequent activation. Such a source may be hydrogen peroxide (H₂O₂) or a precursor species that produces hydroxyl ions. Other sources of hydroxyl ions may be used as appropriate. Examples of other operable sources of hydroxyl ions include peracetic acid (CH₂COO—OH+H₂O), sodium percarbonate (2Na₂CO₃+3H₂O₂), and gluteraldehyde (CH₈O₂). Other activatable species and sources of such other activatable species may also be used.

The cleaning fluid may also contain promoting species that are not themselves sources of activatable species such as hydroxyl ions, but instead modify the sterilization reactions in some beneficial fashion. Examples include ethylenediaminetetraacetate (EDTA), which binds metal ions and allows the activated species to destroy the cell walls more readily; an alcohol such as isopropyl alcohol, which improves wetting of the mist to the cells; enzymes, which speed up or intensity the redox reaction in which the activated species attacks the cell walls; fatty acids, which act as an ancillary anti-microbial and may combine with free radicals to create residual anti-microbial activity; and acids such as citric acid, lactic acid, or oxalic acid, which speed up or intensity the redox reaction and may act as ancillary anti-microbial species to pH-sensitive organisms. Mixtures of the various activatable species and the various promoting species may be used as well. The cleaning fluids are preferably aqueous solutions, but may be solutions in organics such as alcohol. The cleaning fluid source may be a source of the cleaning fluid itself, or a source of a cleaning fluid precursor that chemically reacts or decomposes to produce the cleaning fluid.

As used herein, the term “a nonthermal plasma actuator” or “applicator” means an actuator that activates the cleaning fluid to an activated condition such as the ionized, plasma, or free radical states which, with the passage of time, returns to the non-activated state (a process termed “recombination”). To accomplish the activation, the activator produces activating energy such as electric energy or photonic energy. The photonic energy may be produced by a laser. Examples of activators include an AC electric field, an AC arc, a DC electric field, a pulsed DC electric field, a DC arc, an electron beam, an ion beam, a microwave beam, a radio frequency beam, and an ultraviolet light beam. The activator may include a tuner that tunes the amplitude, frequency, wave form, or other characteristic of the activating energy to achieve a desired, usually a maximum, re-combination time of the activated cleaning fluid mist. As used herein, the term “plasma activated ionic particles” means activated OH⁻ ions.

The term “encircle” as used herein refers to the formation of a continuous circle around an object. The term is not restricted to embodiments of surrounding an entirety of an object or even a major portion of an object. Thus, the phrasing that the system described hereins described herein may be configured such that mist encircles an exterior surface of an apparatus refers to the formation of a continuous ring of mist around at least some exterior portion of the apparatus. In addition, the phrasing that the system described hereins described herein may be configured such that mist propagated to a region encircling an apparatus during an operation of the apparatus collectively occupies the entirety of the encircling region refers to each part of a continuous ring region around an apparatus being exposed to mist at some time during the operation of the apparatus.

The phrase “operating parameter” as used herein refers to any parameter which may affect operation of sterilization with ionized hydrogen peroxide, including but not limited to run time of an applicator, position of an applicator, orientation of components comprising an applicator, dosing parameters for the spray, and/or power supplied to plasma arc. In cases in which the ionized hydrogen peroxide includes a pulsed pattern of spray in application, dosing parameters may include pulse duration and/or pulse frequency.

### A Method for Decontaminating a Closed Space

An aspect of the application is a method for decontaminating a closed space, comprising the steps of: entering input parameters of the closed space into a processing unit, wherein the processing unit is programmed to determine fluid properties of a cleaning fluid in a decontamination device based on the input parameters of the closed space, activating a decontamination cycle of the decontamination device, wherein the decontamination cycle comprises the steps of: providing a reservoir of the cleaning fluid; setting the determined fluid properties of the cleaning fluid; generating a very dry mist comprising cleaning fluid at substantially one atmosphere ambient pressure comprising aerosol droplets, wherein the cleaning fluid comprises a source of an active species for sterilization, wherein the active species is hydroxyl ions and wherein the source is hydrogen peroxide, and the cleaning fluid is silver-free chlorine-free and peracetic-acid free; wherein the mist is subjected to a nonthermal plasma actuator to form plasma activated ionic particles carried by the aerosol droplets of the mist, wherein the plasma activated ionic particles arc hydroxyl ions; wherein a flow meter monitors a flow of the mist through a self-cleaning nozzle into the closed space, and wherein the flow rate of the mist through the self-cleaning nozzle is not greater than 2 ml/min; wherein a pump controls flow rates and pressure through the self-cleaning nozzle by a punp, wherein air pressure within the closed space is maintained as neutral; wherein the self-cleaning nozzle receives the flow of the mist through tubing of not greater than ⅛″ and there is no cavity permitted within the self-cleaning nozzle, wherein the generated very dry mist is applied to decontaminate the closed space.

Methods and technologies preferable for use in sterilization processes are discussed in U.S. Pat. No. 10,391,188, which is incorporated herein by reference. A sterilization fluid mist is activated to produce an activated sterilization fluid mist. The activation produces activated species of the sterilization fluid material in the mist, such as the sterilization fluid material in the ionized, plasma, or free radical states. At least a portion of the activatable species is activated, and in some cases some of the promoting species, if any, is activated. A high yield of activated species is desired to improve the efficiency of the sterilization process, but it is not necessary that all or even a majority of the activatable species achieve the activated state. Any operable activator may be used. The activator field or beam may be electrical or photonic. Examples include an AC electric field, an AC arc, a DC electric field, a DC arc, an electron beam, an ion beam, a microwave beam, a radio frequency beam, and an ultraviolet light beam produced by a laser or other source. The activator causes at least some of the activatable species of the sterilization fluid in the sterilization fluid mist to be excited to the ion, plasma, or free radical state, thereby achieving “activation”. These activated species enter redox reactions with the cell walls of the microbiological organisms, thereby destroying the cells or at least preventing their multiplication and growth. In the case of the preferred hydrogen peroxide, at least some of the H₂O₂ molecules dissociate to produce hydroxyl (OH⁻) and monatomic oxygen (O⁻) ionic activated species. These activated species remain dissociated for a period of time, typically several seconds or longer, during which they attack and destroy the biological microorganisms. The activator is preferably tunable as to the frequency, waveform, amplitude, or other properties of the activation field or beam, so that it may be optimized for achieving a maximum recombination time for action against the biological microorganisms. In the case of hydrogen peroxide, the dissociated activated species recombine to form diatomic oxygen and water, harmless molecules.

Exemplary sterilization devices/systems of the present disclosure comprise an applicator having a cold plasma arc that splits a hydrogen peroxide-based solution into reactive oxygen species, including hydroxyl radicals, that seek, kill, and render pathogens inactive. The activated particles generated by the applicator kill or inactivate a broad spectrum of pathogens and are safe for sensitive equipment. In general, sterilization devices/systems of the present disclosure allow the effective treatment of an exemplary space measuring 104 m2 in about 75 minutes, including application time, contact time, and aeration time. Sterilization devices/systems of the present disclosure are scalable and configurable to be effective in any size or volume of space/chamber/container. The scalability may be accomplished by the size of the device, by the manual control of the sterilization fluid, or by programming the air pressure of the device and the consequent fluid flow rate as a function of the input space/chamber/container parameters.

Conventional methods of sterilization are less effective in sterilizing closed spaces. This application discloses that sterilization using a very dry mist comprising ionized hydrogen peroxide provides unexpectedly high levels of kill rate of pathogens (which encompasses bacteria, fungi, protozoan or viruses), such as, e.g., Candida auris, in closed spaces, semi-enclosed spaces and closed areas (a closed space is an area of 12″×12″×12″ or less; a semi-enclosed space is an area in which part of a closed space is open to other areas; a closed area is an area in which no parts of the closed space are open to other areas).

A very dry mist is a mist in which particles have particle size diameter within the ranges of about 0.1-0.2 microns, 0.1-0.3 microns, 0.1-0.4 microns, 0.1-0.5 microns, 0.1-0.6 microns, 0.1-0.7 microns, 0.1-0.8 microns, 0.1-0.9 microns, 0.1-1 microns, 1-1.1 microns, 1-1.2 microns, 1-1.3 microns, 1-1.4 microns, 1-1.5 microns, 1-1.6 microns, 1-1.7 microns, 1-1.8 microns, 1-1.9 microns, 1-2 microns, 0.5-0.6 microns, 0.5-0.7 microns, 0.5-0.8 microns, 0.5-0.9 microns, 0.5-1 microns, 0.5-1.1 microns, 0.5-1.2 microns, 0.5-1.3 microns, 0.5-1.4 microns, 0.5-1.6 microns, 0.5-1.7 microns, 0.5-1.8 microns, 0.5-1.9 microns, 0.5-2 microns, 0.5-2.1 microns, 0.5-2.2 microns, 0.5-2.3 microns, 0.5-2.4 microns, 0.5-2.5 microns, 0.5-2.6 microns, 0.5-2.7 microns, 0.5-2.8 microns, 0.5-2.9 microns, 0.5-3 microns, 0.5-3.1 microns, 0.5-3.2 microns, 0.5-3.3 microns, 0.5-3.4 microns, or 0.5-3.5 microns. In certain embodiments, the very dry mist has particles with particle diameter size in the range of about 0.5-3 microns, preferably on average 0.7 microns.

In certain embodiments, the system described hereins described herein monitor the size of the aerosol droplets being produced, so that the aerosol droplets carrying activated hydroxyl ions form a very dry mist as described herein. In preferred embodiments, the population of aerosol droplets at least 80%, 90%, 95%, 100% are within the size range of 0.3-1.0 microns in diameter. In particular embodiments, the size of aerosol droplets is monitored by use of laser scanning of aersol droplet size. Optical measurements may be performed with a sensor or a particle detector placed in the detection zone after the point of activation of hydroxyl ions on the aerosol droplets, sensors may be an optical particle counter (OPC), a laser particle counter (LPC), or a condensation particle counter (CPC). OPCs or LPCs can detect particle sizes larger than 0.1 microns. The system described hereins are equipped with a computer processor as described herein, which receives data regarding the size range of aersol droplets carrying activated hydroxyl ions. The system described herein is programmed to adjust control parameters governing the size of particles in the very dry mist to maintain the population of aersol droplet sizes within the desired range.

An aspect of this application discloses the use of system described herein that may be used for sterilization of closed spaces by using a very dry mist comprising ionized hydrogen perodice. The system described herein includes a programming clock, and provides air pressure control and fluid flow control through use of one or more potentiometers. The programming clock provides the ability to automate cycles of sterilization within a closed space. The cycles of sterilization controlled by the programming clock may, for example, include cycles of spraying a very dry mist for thirty seconds, stopping spray for ten seconds, and then re-starting spraying for another thirty seconds, etc, repeating such cycles for a fixed period of time. The programming clock can be set manually by a user or controlled remotely by wireless by the user or a computer processor with pre-programmed sterilization cycles that are transmitted to the device for deployment.

In certain embodiments, the time period during sprayings may be 10-1800 seconds, 10-1200 seconds, 10-900 seconds, 10-600 seconds, 10-300 seconds, 10-180 seconds, 10-150 seconds, 10-120 seconds, 10-90 seconds, 10-60 seconds, 10-45 seconds, 10-30 seconds, 30-1800 seconds, 30-1200 seconds, 30-900 seconds, 30-600 seconds, 30-300 seconds, 30-180 seconds, 30-150 seconds, 30-120 seconds, 30-90 seconds, 30-60 seconds, 30-45 seconds, 60-1800 seconds, 60-1200 seconds, 60-900 seconds, 60-600 seconds, 60-300 seconds, 60-180 seconds, 60-150 seconds, 60-120 seconds, 60-90 seconds, 90-1800 seconds, 90-1200 seconds, 90-900 seconds, 90-600 seconds, 90-300 seconds, 90-180 seconds, 90-150 seconds, 90-120 seconds, 120-1800 seconds, 120-1200 seconds, 120-900 seconds, 120-600 seconds, 120-300 seconds, 120-180 seconds, 120-150 seconds, 150-1800 seconds, 150-1200 seconds, 150-900 seconds, 150-600 seconds, 150-300 seconds, 150-180 seconds, 180-1800 seconds, 180-1200 seconds, 180-900 seconds, 180-600 seconds, 180-300 seconds, 300-1800 seconds, 300-1200 seconds, 300-900 seconds, 300-600 seconds, 600-1800 seconds, 600-1200 seconds, 600-900 seconds, 900-1800 seconds, 900-1200 seconds or 1200-1800 seconds.

In certain embodiments, the time period between two consequent sprayings may be 1-600 seconds, 1-300 seconds, 1-180 seconds, 1-150 seconds, 1-120 seconds, 1-90 seconds, 1-60 seconds, 1-45 seconds, 1-30 seconds, 1-15 seconds, 10-600 seconds, 10-300 seconds, 10-180 seconds, 10-150 seconds, 10-120 seconds, 10-90 seconds, 10-60 seconds, 10-45 seconds, 10-30 seconds, 30-600 seconds, 30-300 seconds, 30-180 seconds, 30-150 seconds, 30-120 seconds, 30-90 seconds, 30-60 seconds, 30-45 seconds, 60-600 seconds, 60-300 seconds, 60-180 seconds, 60-150 seconds, 60-120 seconds, 60-90 seconds, 90-600 seconds, 90-300 seconds, 90-180 seconds, 90-150 seconds, 90-120 seconds, 120-600 seconds, 120-300 seconds, 120-180 seconds, 120-150 seconds, 150-600 seconds, 150-300 seconds, 150-180 seconds, 180-600 seconds, 180-300 seconds, or 300-600 seconds. In one example, the time period between two consequent sprayings is 60 seconds.

In some cases, the time period during spraying is 90 seconds, with 60 second intervals between spraying. In some embodiments, a spray circle comprises a spray time and a break time, and a complete sterilization process comprises 1, 2, 3, 4, 5, 6, 7, 8, 9 or 10 spray circles. (Spray circle=spray+interval−total number of circles.)

In certain embodiments, the system described herein will possess a computer processor that can calculate the appropriate settings (e.g. flow rate, air pressure, number and length of sterilization cycles) to produce a very dry mist comprising ionized hydrogen peroxide that will effectively decontaminate a closed space. In such embodiments, the user may enter the parameters of the closed space manually to the device, or enter them remotely by a wireless connection. The operation of the device can be fully automated, fully remotely controlled, or may be semi-automated (e.g., uses cycles of sterilization performed automatically according to parameters that have been manually entered).

It is a common problem of the conventional technology that excessive air pressure reduction produces mist particles that are too large to achieve a desired mist/fog profile. At the same time, particularly closed space spaces often require significant air pressure reduction. These opposing constraints of a sterilization system are addressed by certain embodiments of the present disclosure. Namely, by programming the processor to control the potentiometer based on the input parameters of the closed space, a user can regulate a fluid flow rate in synchronization with the air pressure. As a result, reducing the fluid flow rate while simultaneously lowering the air pressure maintains the mist/fog particle size small, while limiting the distance the spray can reach. In this manner, the mist sprayed by the system described herein remains within the boundaries of the closed space, without creating excessively wet and dense fog. The programmable balance between the air pressure and the fluid flow rate, therefore, prevents saturating surfaces opposite to mist applicators, increased moisture accumulation due to condensation, false negative validation results or increased aeration times of the closed space.

The present application describes methods, systems, and means of disinfection through use of automated devices that effectively apply a mist with activated hydroxyl ions in which aerosol droplets will produce an effective high surface area of activated hydroxyl ions. It is an advantage of this approach that no chemical residue is left behind on the disinfected surface, because starting with a small quantity of hydrogen peroxide as a source solution, and then activating hydroxyl ions, means that the dissociated activated species recombine to form diatomic oxygen and water, which are harmless molecules.

Effective disinfection by activated hydroxyl ions as used in the present application depends on the surface area of the droplets that are applied to surfaces; that is the smaller the droplets, the greater the surface area of activated hydroxyl ions on the total cloud of droplets, and thus, the more effective the disinfection method. In fact, soaking a surface for disinfection undermines effectiveness of activated hydroxyl ions because once a surface is soaked the activated ions will not be brought into contact with the bacteria for disinfection.

Activation of the cleaning fluid to produce activated hydroxyl ions may occur through passage of the fluid, for example, an electric arc current, an electromagnetic field, or photonic energy. The fluid may be generated as a spray via, for example, nebulization, ultrasonices, pneumatic spray, or mechanical pressure. However, blowers are not used in the method of the application to generate a spray, as a blower will generate a powerful stream of large droplets that will soak a surface with fluid, which both undermines the impact of any activated hydroxyl ions.

The methods of the application require that a very dry mist (very low diameter aerosol particles as described herein) be generated which carries activated hydroxyl ions through a space to a surface for sterilization. The activated hydroxyl ions make contact with pathogens before recombining to form harmless diatomic oxygen and water (it is an advantage of the approach herein that no chemical residue remains on the disinfected surface). Preferred embodiments of the present application use, for example, a cleaning fluid that comprises 0.3% to 9% hydrogen peroxide as a source of an active species for sterilization of an article or cell processing device. Preferred aerosol droplets that carry activated hydroxyl ions are 0.3-1.0 microns in diameter, with most preferred to average 0.7 microns in diameter. Accordingly, any automated systems applying the present methods require exacting parameters for performance.

In a certain embodiment, a cell processing system includes a closed space. Closed space may comprise an enclosure having four walls, a base, and a roof. The closed space may be divided into an interior zone with a feedthrough access, and quality control (QC) instrumentation. An air filtration inlet (not shown) may provide high-efficiency particulate air (HEPA) filtration to provide ISO7 or better air quality in the interior zone. This air filtration may maintain sterile cell processing in an ISO8 or ISO9 manufacturing environment. The closed space may also have an air filter on the air outlet to preserve the ISO rating of the room. In some variations, the closed space may further comprise, inside the interior zone, a bioreactor instrument, a cell selection instrument (e.g., MACS), an electroporation instrument (EP), a counterflow centrifugation clutriation (CCE) instrument, a sterile liquid transfer instrument (e.g., fluid connector), a reagent vault, and a sterilization system. The reagent vault may be accessible by a user through a sample pickup port. A robot (e.g., support arm, robotic arm) may be configured to move one or more cartridges (e.g., consumables) from any instrument to any other instrument and/or move one or more cartridges to and from a reagent vault. In some variations, the closed space may comprise one or more moveable barrier (e.g., access, door) configured to facilitate access to one or more of the instruments in the closed space.

In some variations of methods according to the disclosure, a human operator may load one or more empty cartridges into the feedthrough via cartridge port. The cartridges may be pre-sterilized, or the feedthrough may sterilize the cartridge using ultraviolet radiation (UV), or chemical sterilizing agents provided as a vapor, spray, or wash. The feedthrough chamber may optionally be configured to decontaminate cartridges with activated hydroxyl radicals as described herein to maintain sterility of the interior zone (e.g., ISO 7 or better). The cartridge may be passed to the biosafety system described herein, where input cell product is provided and loaded to the cartridge through a sterile liquid transfer port into the cartridge. The user (via robot) may then move the cartridge back to the feedthrough and initiate automated processing using a computer processor in the computer server rack (e.g., controller). The robot may be configured to move the cartridge in a predefined sequence to a plurality of instruments and stations, with the components of the closed space. At the end of cell processing, the cartridge, now containing the processed cell product, may be returned to the feedthrough for retrieval by the user. In some variations, an outer surface of the enclosure may comprise an input/output device (e.g., display, touchscreen).

In some variations, one or more components of a sterilization system (e.g., sterilant source, pump) may be coupled to a closed space. For example, a cell processing system comprising a closed space, sterilization system, fluid connector and fluid devices. In some variations, the fluid devices may comprise a main (e.g., consumable) feedthrough and a fluid device (e.g., reagent) feedthrough. The sterilization system may comprise a sterilant source, pump, and heater (e.g., desiccant/dryer). For example, the heater may be configured to acrate at a predetermined set of conditions. The sterilization system may be coupled and in fluid communication with one or more of the closed space, fluid connector, and fluid device. In some variations, a robot (not shown) may be configured to manipulate and operate the cell processing system. For example, the fluid connector may be coupled to one or more of the fluid devices and instruments (not shown). One or more of the closed space, fluid connector, and fluid devices may be sterilized and/or acrated by circulating one or more of a sterilant comprising the activated hydroxyl radicals as described herein using the sterilization system. In some variations, the sterilization system may provide a sterility assurance level (SAL) of at least 10−3 SAL.

Generally, sterilization of a fluid connector may comprise one or more steps of dehumidification, conditioning, sterilization, and aeration (e.g., ventilation). Dehumidification may include removing moisture from the fluid connector. Conditioning may include heating the surfaces of the fluid connector to be decontaminated in order to prevent condensation and aid sterilization. Sterilization may include circulating a sterilant through the fluid connector at a predetermined concentration, rate, and exposure time. Acration may include removing the sterilant from the fluid connector by circulating a gas (e.g., sterile air) through the fluid connector.

A sterilant may be flowed into the fluid connector to sterilize one or more portions of the fluid connector. As described in more detail herein, the sterilant comprises activated hydroxyl radicals as described herein. The method may comprise flowing a sterilant into the fluid connector through the sterilant port. The fluid connector has a first chamber, where the first chamber receives the sterilant for a predetermined amount of time (e.g., dwell time). For example, the sterilant may be circulated within the chamber to sterilize the chamber of the fluid connector and any contents disposed therein (e.g., other fluid, biological material). In some variations, the dwell time may be up to about 10 minutes, and between about 1 minute to about 10 minutes, including all ranges and sub-values in-between. Additionally or alternatively, one or more of the first valve and the second valve may be in the open configuration such that the sterilant may be circulated through other portions of the fluid connector such as first proximal end and second proximal end.

### A System for Decontaminating a Closed Space

An aspect of the application is system for decontaminating a closed space, comprising a decontamination device and a computer processor, wherein the computer processor is in networked communication with the decontamination device, wherein input parameters of the closed space are entered into the computer processor, wherein the computer processor is programmed to determine fluid properties of a cleaning fluid in the decontamination device based on the input parameters of the closed space, wherein the computer processor is further programmed to activate a decontamination cycle of the decontamination device, the decontamination cycle comprising the steps of: providing a reservoir of the cleaning fluid; setting the determined fluid properties of the cleaning fluid; generating a very dry mist comprising cleaning fluid at substantially one atmosphere ambient pressure comprising aerosol droplets, wherein the cleaning fluid comprises a source of an active species for sterilization, wherein the active species is hydroxyl ions and wherein the source is hydrogen peroxide, and the cleaning fluid is silver-free chlorine-free and peracetic-acid free; wherein the mist is subjected to a nonthermal plasma actuator to form plasma activated ionic particles carried by the aerosol droplets of the mist, wherein the plasma activated ionic particles are hydroxyl ions; wherein a flow meter monitors a flow of the mist through a self-cleaning nozzle into the closed space, and wherein the flow rate of the mist through the self-cleaning nozzle is not greater than 2 ml/min; wherein a pump controls flow rates and pressure through the self-cleaning nozzle by a pump, wherein air pressure within the closed space is maintained as neutral; wherein the self-cleaning nozzle receives the flow of the mist through tubing of not greater than ⅛″ and there is no cavity permitted within the self-cleaning nozzle, wherein the generated very dry mist is applied to decontaminate the closed space.

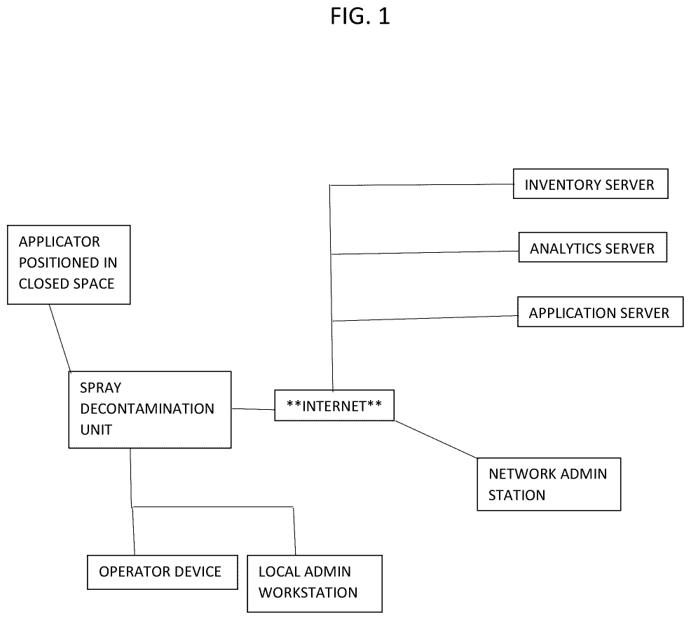

Herein, a control system is a computer-implemented control system that can deliver sterilization processes using various applicators, FIG. 1 shows an embodiment of the architecture of the system components and their relationships. In this embodiment, the control system utilizes a general-purpose computer. The control system is operated by the general-purpose computer. The general-purpose computer does not require specialized hardware and provides the greatest flexibility of programming languages, development environments, and software and accessory support. Furthermore, a general-purpose computer provides reliable I/O, an easily installable operating environment, and lowest cost.

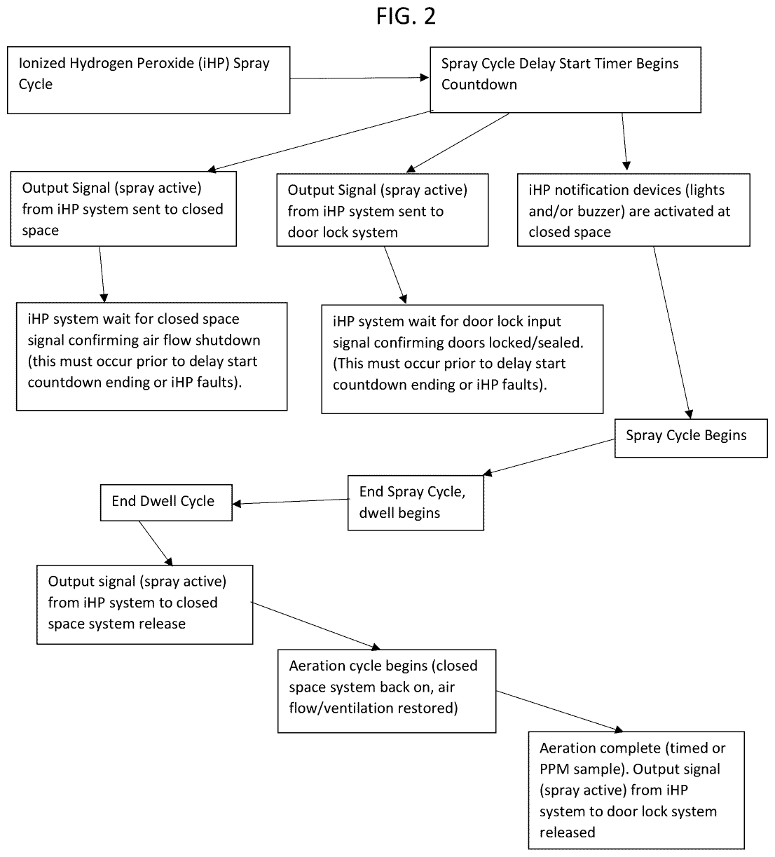

An embodiment of the flow of the sterilization system is shown in FIG. 2. The user initiates the start of the cycle-system delays the start of the system for the sterilization to be shutdown (either manually or by signal sent from system). As can be seen in FIG. 2, the ionized hydrogen peroxide (iHP) spray cycle begins when the start button is pressed (this may be done through the networked systems described herein via a user interface). Following pressing the start button, a spray cycle delay start timer begins its countdown. This enables any individuals in areas to vacate them before the decontamination begins. It also gives time for individuals to leave before doors are locked so that areas for decontamination are sealed. The system may trigger warning alarms that make alarm sounds to alert individuals in the areas targeted for decontamination and/or verbal warnings that are heard within areas for decontamination, including, for example, a verbal countdown. Notification devices, such as lights or buzzers, may be present on applicators which are located within areas for decontamination; these notification devices will start flashing or buzzing to alert individuals within the areas to leave. An output signal is sent from the sterilization system to the sterilization system to alert the sterilization system that the spray is active; however, the ionized hydrogen peroxide spray will not begin at this point, instead the system will wait for the sterilization system to signal the air flow has shutdown in the targeted areas for decontamination. The delay start countdown does not end until after the sterilization system has received an input signal from the sterilization system that air flow has shutdown. An output signal is also sent from the sterilization system to the device lock system; the system waits for a device lock input signal that confirms that all device doors are locked before ending the delay start countdown.

The spray cycle begins once the system has received notification that all doors have been sealed and airflow has been shutdown. The customized engineered system monitors the amount of solution requested both in flow rate during injection, and total amount delivered (system will fault if the system detects a loss of flow from any applicator-if more than one applicator is installed in a treatment area the system can be programed either to shut the cycle or allow the remaining applicator(s) to continue until the correct required amount of solution has been delivered).

The spray cycle ends based on the programmed parameters, and then the dwell cycle begins. Dwell commences after the injection cycle (this pause period is timed and is adjustable by customer). During the dwell cycle the customized engineered system is in standby, treatment area(s) remain under the control of the customized engineered system.

Once the dwell cycle ends, an output signal is sent from the sterilization system to the sterilization system release. After dwell, aeration commences. The customized engineered system will signal the sterilization/exhaust system to turn on (if equipped).

After the sterilization system is released an aeration cycle begins in which the sterilization system is back on and air flow/ventilation restored. The aeration cycle proceeds to remove the residue of the ionized hydrogen peroxide decontamination (diatomic oxygen and water as described herein) from the device on either a timed based is based on measuring parts per million (PPM) air samples (or both). Once the aeration cycle is complete, an output signal is sent from the sterilization system to the door lock system to be released. The system monitors the treatment area(s) exhaust for residual concentrations of solution (all doors remain locked until a preset safe level is achieved) or the system can be set to release door(s) upon a preset time.

The system may be equipped with cameras and the ability to view through the cameras the areas that are to be decontaminated; the camera views may either be assessed by a user or by an algorithm designed to recognize human or animal movement in the area for decontamination, if the algorithm recognizes such movement that the spray cycle will fault until the issues is resolved.

Air CDA is also monitored during the injection cycle for a lost at each applicator and at the inlet of the air supply. The arc is also monitored at each applicator. An air monitor is placed in appropriate locations to view monitoring of PPM level. If each area being decontaminated has a separate exhaust and has the possibility of being operated individually then individual low-level monitors can be used for each treatment area. The monitors will monitor the peak PPM at the start of the aeration cycle to provide a data set point (providing a range from maximum to minimum PPM levels).

Upon a completed cycle system can send information to the users server for distribution as user deems necessary. Upon a faulted cycle system can send information to users server for distribution as user deems necessary

In an exemplary embodiment, the computer system includes a memory, a processor, and, optionally, a secondary storage system described herein. In some embodiments, the computer system includes a plurality of processors and is configured as a plurality of, e.g., bladed servers, or other known server configurations. In particular embodiments, the computer system also includes an input device, a display device, and an output device. In some embodiments, the memory includes RAM or similar types of memory. In particular embodiments, the memory stores one or more applications for execution by the processor. In some embodiments, the secondary storage device includes a hard disk drive, floppy disk drive, CD-ROM or DVD drive, or other types of non-volatile data storage. In particular embodiments, the processor executes the application(s) that are stored in the memory or the secondary storage, or received from the internet or other network. In some embodiments, processing by the processor may be implemented in software, such as software modules, for execution by computers or other machines. These applications preferably include instructions executable to perform the functions and methods described above and illustrated in the Figures herein. The applications preferably provide GUIs through which users may view and interact with the application(s). In other embodiments, the system comprises remote access to control and/or view the system.

A still further aspect of the application is a non-transitory computer readable medium within a CPU of the system described herein providing instructions for repeating sterilization cycles of a sterilization apparatus, the instructions comprising: sensing a presence of a pathogen in a cell processing device; communicating the presence of the pathogen to a computer database; identifying the pathogen sensed in the cell processing device using the computer database; selecting a program of sterilization cycles from the computer database based on the identity of the pathogen; communication the selected program to a sterilization apparatus, wherein the sterilization apparatus is networked to automatically follow the program; performing the sterilization cycles according to the program, wherein each sterilization cycle comprises the steps of: providing a reservoir of a cleaning fluid; cavitating the reservoir of cleaning fluid by applying force to the cleaning fluid; generating a mist comprising aerosol droplets, wherein the mist is generated from the cleaning fluid while the cleaning fluid is subject to cavitation by force; subjecting the mist to a nonthermal plasma actuator to form plasma activated ionic particles; and contacting the plasma activated ionic particles to the airborne pathogen.

In some embodiments, the sterilization system described herein further contains a control module that allows control (e.g., start and or stop the system described herein) and monitoring of the miniature sterilizing system described herein from a remote system described herein such as a tablet or a phone. In some embodiments, the control module further controls data storage, transfer and printing. In some embodiments, the control module allows for remote service and connection, for recording video or data, and for providing feedback to the manufacture during use or after use.

In still other embodiments, the voltage source can be switched between AC and DC. The mist generator is functionally connected to a mist delivery unit which may be mounted on the housing or is a remote unit. In some embodiments, the mist delivery unit is hand held, mounted on another apparatus, or held by/mounted on another machine or a system described herein. In some further embodiments, the system described hereins are self-navigating and patrol an area. In some embodiments, the voltage source is AC. In other embodiments, the voltage source is DC. In still other embodiments, the voltage source can be switched between AC and DC. In particular embodiments, the mist is dispersed from the unit via high voltage actuation. In some embodiments, the high voltage actuation is persistent. In other embodiments, the high voltage actuation is intermittent. In particular embodiments, the high voltage actuation charges the mist and further atomizes the droplets.

In some embodiments, the sterilization system described herein comprises an ultrasonic wafer or ultrasonic nebulizer as a mist generator. In some embodiments, the mist generator comprises a substantially closed sonication chamber that comprises a bottom chamber portion or reservoir, a top chamber portion forming a pathway between the bottom chamber portion and a plasma actuator, a voltage source, a side chamber portion comprising a cleaning fluid source and an interior chamber portion, wherein the cleaning fluid 80 that is dispensed into the nebulizer is sheared by ultrasonic cavitation generated by a ultrasonic cavitation system described herein within the sonication chamber. The cleaning fluid is introduced into a fluid chamber or reservoir until it submerges an ultrasonic cavitator. The ultrasonic cavitator produces resonant ultrasonic waves that serve to cavitate the cleaning fluid, which produces a mist of aerosol droplets that rise from the fluid through a pathway. The mist passes through an applicator head and a plasma actuator, or electrodes, where the particles are activated before entering the external atmosphere. In some embodiments a fan may be used to direct the flow of the mist. In certain embodiments, the system described herein comprises a rotating applicator based with a small circulating fan. In other embodiments, the system described herein comprises a self-contained applicator that would include air compressor, fluid pump, and transformer. In some embodiments, heating elements heat the space inside to spread the nebulized mist. In some embodiments, the system described herein comprises rotating heads or self-cleaning nozzles.

The pathway can take any form suitable to direct the aerosol droplets from the reservoir to the plasma actuator. In some embodiments, the pathway is in the form of a funnel. In other embodiments, the pathway may be, but is not limited to, in the form of a pipe, tube, elbow or cylinder.

In some embodiments, the plasma actuator is nonthermal. In other embodiments, the plasma actuator is thermal.

In certain embodiments, a mobile/wireless/remote control system described herein is functionally connected to a sterilization system described herein of the present disclosure. The functional connection can be wired or wireless. In some embodiments, a wireless connection includes, but is not limited to, radio frequency, infrared, wifi, BLUETOOTH, or any other suitable means of wireless communication. In some embodiments, the control device sends control instructions to the system described herein via the functional connection and the system described herein sends feedback data to the control system described herein via the functional connection.

In certain embodiments, a system wherein a mist generator, cleaning fluid source and mist delivery unit are further interfaced with a sensor. In some embodiments, the sensor detects microbes (such as bacteria, protozoan, parasites, amocbac, or viral particles), that are airborne or contaminating a surface. In some embodiments, the sensor, upon detection of contaminants, automatically triggers actuation of the system.

### Operating Parameters for Sterilization System

In some embodiments, the system described herein may include or may be configured to access a database listing characteristics in which the system described herein is deployed. In addition or alternatively, the system described herein may include a system for collecting and/or generating data regarding characteristics of a cell population for which the system described herein is deployed. In such cases, any system known in the art for collecting, generating and/or analyzing characteristics of a cell population may be used, depending on the data to be generated. Examples include spatial sensors, photo recognition systems and/or dosimeters. A system may, in some embodiments, be operationally coupled to a CPU. Alternatively, a CPU may be configured to access cell population characteristic data from a database. In either case, CPU may be configured to retrieve and access data regarding characteristics of the cell population for which the system described herein is deployed and determine an operating parameter of the applicator for application of ionized hydrogen peroxide, such as a position of the applicator based on the data. In some embodiments, the determined operating parameter may be relayed via a user interface such that a user of the system described herein may be informed to invoke the operating parameter for the system described herein, such as movement to a particular position. In other cases, a CPU may be configured to send a command in accordance with the determined operating parameter to a means within the system described herein for automatically invoking the operating parameter, such as automatically moving the direction of spray of the applicator.

In some embodiments, a system may be used to measure doses of ionized hydrogen peroxide received at an object or spot in a chamber in which the system described herein is deployed. In particular, measuring the dose of ionized hydrogen peroxide at an object or spot in a chamber may aid in determining operating parameter of the applicator, such as optimizing the placement of the applicator. As noted above, one of the primary factors affecting ionized hydrogen peroxide effectiveness on an object is distance to the object. Through the operational coupling of system to a CPU, the CPU may be configured to retrieve measurements from the system, determine an operating parameter of the applicator based on the measurements, such as a position of the applicator, and cither relay the determined operating parameter to a user interface and/or send a command in accordance with the determined operating parameter to a means within the system described herein for automatically invoking the operating parameter, such as an applicator. In general, any system known in the art for measuring spray doses may be used for the system.

The system described herein may include or may be configured to access a database listing characteristics of one or more cell populations and/or apparatus may include a system for collecting and/or generating data regarding characteristics of a cell population. Any system known in the art for generating, collecting and/or analyzing characteristics of a cell population may be used. Examples include dosimeters, spatial sensors and/or photo recognition systems. In some cases, apparatus may further include a CPU to retrieve data, determine a position of the applicator based on the data, and either relay the determined position to a user interface and/or send a command in accordance with the determined position to a means within the system described herein for automatically moving the applicator.

In certain embodiments, the system described herein comprises laser diffraction technology to quantify particle size distribution in in the spray. The applicator is mounted outside in ambient air and the spray travels through an enclosed self-cleaning nozzle. The spray is formed by ultrasonics, or similar means, generated within a chamber within the system described herein and the spray flows through the applicator to the outer air. Midway through the self-cleaning nozzle, the spray travels through the zone in which the laser is projected thus causing the laser beam to diffract after colliding with the particles. A collection of sensors across from the laser source measure these diffraction patterns and, using Mic theory (an analytical solution of equations for the scattering of electromagnetic radiation by spherical particles), interprets them to quantify particle size distribution of the ionized particles in the spray. Since the method herein relies on application of a very dry mist, the system described herein may be programmed to adjust air valve, or other operating parameters, to maintain suitably low mean diameter of ionized particles as determined by laser diffraction technology.

In some embodiments disclosed herein, a sterilization system described herein includes a microprocessor connected to a memory and a wireless network circuit, for executing routines stored in the memory and commands generated by the routines and received via a wireless network circuit. The sterilization system described herein includes driven wheels commandable by the microprocessor to reach a multiplicity of accessible two dimensional locations within a space, and at least one applicator, commandable by the microprocessor to perform sterilization using ionized hydrogen peroxide within the space, the microprocessor executing a plurality of routines. The plurality of routines include a first routine which monitors a wireless local network by communicating with the wireless network circuit, and detects a presence state of one or more sterilization system described hereins on the wireless local network, a second routine which receives a signal from a sensor in the sterilization system described herein, the sensor detecting a pathogen within the space, and a third routine which commands the system described herein to change state of performing sterilization based on the presence of the pathogen within the space and the location and activities of other system described hereins on the wireless local network.

Network entities, such as thermostats, air purifiers and humidifiers, are stationary and typically located at one or two locations throughout a living space and the stationary sensors therein measure relatively localized air currents at that particular singular, unchanging location. A primary advantage of the sterilization system described herein is accessing locations distant from or in another compartment not immediately adjacent the network entities. By mapping measurements throughout a space, the sterilization system described herein determines whether, based on a single reading taken in a location remote from the network entity or based on an aggregate of readings taken randomly or systematically throughout the space, a stationary network entity should be activated or whether the sterilization system described herein itself should deploy. Based on a plurality of measurements taken throughout a space, the sterilization system described herein activates itself when the environmental sensor reading of the network entities and/or the system described herein indicates that the system described herein should deploy to a point of location of a detected pathogen. The network entities dedicated sensor measures only the immediate volume adjacent the network entity and fails to account for variations in a volume of air mass spread throughout a living space. By monitoring and measuring temperature, air quality and humidity throughout the space, the sterilization system described herein provides information otherwise inaccessible by the network entity.

In some embodiments, the at least one measurable characteristic is time to complete cleaning of a fluid connector and the sensor is a clock. In some embodiments, the at least one measurable characteristic is mission completion and the sensor is a pathogen detecting sensor for airborne pathogen or surface pathogens within the fluid connector based on the score of the pathogen detection measurement in the fluid connector.

### Power Specification for Sterilization System

Another aspect of the present application relates to sterilization system described herein that comprise a DCV miniature transformer and/or a DCV miniature compressor to reduce power demand and overall weight and size of the system described herein. In certain embodiments, the system described herein comprises a small chamber system that heats the sterilizing solution to cause vaporization before passing through the arc system. In particular embodiments, the system described herein comprises a rechargeable battery operated portable wheeled system (similar in form to an IV stand-type system).

In some embodiments, the DCV miniature transformer has an input DC voltage in the range of 6-36V and generates an output of 12-22.5 kV. In some embodiments, the DCV miniature transformer has an input DC voltage of 24V and generates an output of 17.5 kV.

In some embodiments, the DCV miniature compressor provides a pressure in the range of 10-60 psi and has an input DC voltage in the range of 6-36V. In some embodiments, the DCV miniature compressor provides a pressure in the range of 30-40 psi and has an input DC voltage of 24V.

In some embodiments, the miniature sterilization system described herein further comprises a diode/capacitor rectifier that smooth's out arc converting process and increases the converting efficiency in AC.

In some embodiments, the miniature sterilization system described herein further comprises low flow pump with a flow rate in the range of 4-40 ml/min and an operating voltage in the range of 6-36 VDC.

In some embodiments, the miniature sterilization system described herein further contains a control module that allows control (e.g., start and or stop the system described herein) and monitoring of the miniature sterilizing system described herein from a remote system described herein such as a tablet or a phone. In some embodiments, the control module further controls data storage, transfer and printing.

Another aspect of the present application relates to a miniature sterilization system described herein that comprises a miniature transformer and an ultrasonic wafer or ultrasonic nebulizer as a mist generator. In some embodiments, the mist generator comprises a substantially closed sonication chamber that comprises a funnel shaped top chamber portion, a bottom chamber portion, a side chamber portion and an interior chamber portion, wherein the cleaning fluid is sheared by ultrasonic cavitation within the sonication chamber. In some embodiments, the system described herein comprises more than one ultrasonic wafer. In some further embodiments, the system described herein comprises 2, 3, 4, 5, 6, 7, 8, 9, or 10 ultrasonic wafers.

Another aspect of the present application relates to a sterilization system described herein that comprises a diode/capacitor rectifier that smooth's out arc converting process and increases the converting efficiency.

In some embodiments, the sterilization system described herein has a modular structure that reduces the footprint of the system described herein and allows exchange of modules between different system described hereins.

In some embodiments, the sterilization system described herein further comprises low flow pump with a flow rate in the range of 4-40 ml/min and an operating voltage in the range of 6-36 VDC or 10-28 VDC.

In certain embodiments, an aspect of the present application relates to a sterilization system described herein is an autonomous, machine-based solution that provides a more precise and efficient disinfection and sterilization, eliminating potential human error and reducing a facility's operating expenses. It incorporates automated guided vehicle (AGV) technology for intuitive maneuverability in unmapped environments. The software allows for enhanced AI capability to optimize and execute mapping, autonomous navigation, and disinfection routines based on proprietary algorithms. The software also ensures the efficacy of ionized hydrogen peroxide disinfection under optimal operating parameters directed by the user.

In certain embodiments, the system further comprises one or more computing devices, each device having one ore more computer processors, a data communication connection, and one or more tangible non-transitory computer-readable media accessible by the one or more computer processors, and a plurality of databases, including a pathogen database and a pathogen response protocol database, wherein the pathogen database and a pathogen response protocol database are each stored in the one or more tangible non-transitory computer readable media, wherein the one or more tangible non-transitory computer readable media comprise instructions that, when executed by the one or more computer processors, cause the one or more computer processors to perform the steps of: (a) receiving, via the sensors for surface moisture the level of moisture on surfaces within the enclosed space or area infected with a pathogen before the mist generator has sheared the cleaning fluid into a mist; (b) initiating a pathogen response protocol, wherein the aerosol droplets of the mist carrying hydroxyl ions are contacted with surfaces in the enclosed space or area infected with a pathogen for a cycle of time in accordance with the pathogen response protocol; (c) receiving, via the sensors for surface moisture the level of moisture on surfaces within the enclosed space or area infected with a pathogen after the aerosol droplets of the mist carrying hydroxyl ions are contacted with surfaces in the enclosed space or area infected with a pathogen; (d) determining in accordance with the pathogen response protocol whether or not to apply an additional cycle of the aerosol droplets of the mist carrying hydroxyl ions are contacted with surfaces in the enclosed space or area infected with a pathogen; (e) applying another cycle of the aerosol droplets of the mist carrying hydroxyl ions are contacted with surfaces in the enclosed space or area infected with a pathogen if the protocol requires doing so: (f) completing the pathogen response protocol, wherein once the pathogen response protocol is completed the mist is no longer generated; (h) receiving, via the sensors for surface moisture the level of moisture on surfaces within the enclosed space or area infected with a pathogen after the pathogen response protocol has been completed; (i) comparing the surface moisture levels before and after completion of the pathogen response protocol; (j) determining via the sensors for surface moisture when the moisture levels have returned to the levels before initiating generation of the mist.

An aspect of the application is directed to a method for sterilization using a system as described herein for ionized hydrogen peroxide, comprising the steps of: entering input parameters of the space into a processing unit of the system, wherein the processing unit is programmed to determine fluid properties of a sterilization fluid in an ionization/acrosolization and activation device mounted on the system based on the input parameters of the space, activating a sterilization cycle of the ionization/aerosolization and activation device, wherein the sterilization cycle comprises the steps of: providing a reservoir of the sterilization fluid; setting the determined fluid properties of the sterilization fluid; generating a very dry mist comprising ionized hydrogen peroxide of the sterilization fluid, wherein an ionized/aerosolized mist of hydrogen peroxide is passed through a cold plasma arc, and wherein the generated very dry mist is applied to decontaminate the surrounding space.

In certain embodiments, the ionization/aerosolization and activation device is operated remotely. In particular embodiments, the ionization/aerosolization and activation device is operated automatically.

In certain embodiments, the input parameters comprise: dimensions of the space, a position of the ionization/aerosolization and activation device relative to the space, air temperature, pressure, and humidity of the space. In particular embodiments, the set fluid properties of the sterilization fluid comprise air pressure and fluid flow rate. In other embodiments, the setting of the determined fluid properties to the sterilization fluid is performed by controlling an air valve. In certain embodiments, the air valve is controlled by programming the processing unit to control a potentiometer. In various embodiments, the determined fluid properties of the sterilization fluid are adjusted by a size and a shape of a tube located at an exit of the sterilization fluid out of the ionization/acrosolization and activation device.

In particular embodiments, the fluid properties of the sterilization fluid are set by lowering the air pressure and the fluid flow rate respectively below a predetermined standard air pressure and a predetermined standard fluid flow rate.

In particular embodiments, the processing unit and the ionization/aerosolization and activation device are in wireless communication.

Another aspect of the application is a system for sterilization using a system of delivery for ionized hydrogen peroxide, comprising a system as described herein, which comprises an ionization/aerosolization and activation device and a computer processor, wherein the computer processor is in networked communication with the ionization/aerosolization and activation device, wherein input parameters are entered into the computer processor, wherein the computer processor is programmed to determine fluid properties of a sterilization fluid in the ionization/aerosolization and activation device based on the input parameters, wherein the computer processor is further programmed to activate a sterilization cycle of the ionization/aerosolization and activation device, the sterilization cycle comprising the steps of: providing a reservoir of the sterilization fluid; setting the determined fluid properties of the sterilization fluid; generating a very dry mist of the sterilization fluid, wherein an ionized/acrosolized mist of hydrogen peroxide is passed through a cold plasma arc, and wherein the generated ionized very dry mist is applied to decontaminate the space.

In some embodiments, the cleaning fluid comprises hydrogen peroxide at a concentration between 6 to 9%. In particular embodiments, the cleaning fluid comprises hydrogen peroxide at a concentration of about 7.8%. In some embodiments, the article or substantially closed space is exposed to the plasma activated ionic particles in an amount sufficient to provide greater than 6-log 10 reduction of viable bacteria or viable bacterial spores relative to untreated controls. In particular embodiments, the article, surface, or substantially closed space is exposed to the plasma activated ionic particles in an amount sufficient to provide greater than 7-log 10 killing of bacteria or bacterial spores relative to untreated controls. In other embodiments, the article, surface, or substantially closed space is exposed to the plasma activated ionic particles in an amount sufficient to provide greater than 8−log¹⁰ killing of bacteria or bacterial spores relative to untreated controls. In another embodiment, the article, surface, or substantially closed space is exposed to the plasma activated ionic particles in an amount sufficient to provide greater than 9−log¹⁰ killing of bacteria or bacterial spores relative to untreated controls.

In certain embodiments, the power source comprises a DC power source, a high frequency AC power source, an RF power source, a microwave power source, a pulsed DC power source and a pulsed AC power source.

A further aspect of the application is a method for sterilizing a cell processing device using a system as described herein, comprising the steps of: deploying a system described herein within the space to be decontaminated, submerging within a chamber within the system described herein an ultrasonic cavitator in a reservoir of a cleaning fluid; cavitating the cleaning fluid with ultrasonic vibrations produced by the ultrasonic cavitator; generating a mist comprising aerosol droplets, wherein the mist is generated from the cleaning fluid while the cleaning fluid is being cavitated; subjecting the mist to a nonthermal plasma actuator to form plasma activated ionic particles; and contacting the plasma activated ionic particles to a pathogen. In particular embodiments, the pathogen is a bacteria.

A further aspect of the application is a method for sterilizing a cell processing device using a system described herein, comprising the steps of: sensing a presence of an airborne pathogen in the atmosphere of a cell processing device using a sensor mounted on the system described herein; communicating the presence of the airborne pathogen from the sensor to a networked computer processor within the system; communicating from the networked computer processor to a sterilization apparatus that an airborne pathogen is present in the cell processing device; activating a sterilization cycle of the sterilization apparatus, wherein the sterilization cycle comprises the steps of: providing a reservoir of a cleaning fluid; cavitating the reservoir of cleaning fluid by applying force to the cleaning fluid; generating a mist comprising aerosol droplets, wherein the mist is generated from the cleaning fluid while the cleaning fluid is subject to cavitation by force; subjecting the mist to a nonthermal plasma actuator to form plasma activated ionic particles; and contacting the plasma activated ionic particles to the airborne pathogen.

A further aspect of the application is a system for sterilizing a cell processing device using a system described herein, comprising: a sensor within the system described herein for detecting airborne pathogens, wherein the sensor is in networked communication with a computer processor; a computer processor, wherein the computer processor is in networked communication with the sensor and a sterilization apparatus comprised within the system described herein; a sterilization apparatus, wherein the sterilization apparatus is in networked communication with the computer processor, and further wherein the sterilization apparatus comprises: a reservoir of cleaning fluid; an ultrasonic cavitator, wherein the ultrasonic cavitator is submerged in the reservoir; a nonthermal plasma actuator, wherein the actuator activates a mist generated from the reservoir; a funnel, wherein the funnel connects the nonthermal plasma activator to the reservoir; an outer tube, wherein the outer tube connects the nonthermal actuator to the external atmosphere; and wherein a mist generated from the reservoir can pass through the funnel to the actuator, and further wherein after the mist is activated by the actuator the mist can pass through the outer tube to the external atmosphere.

Another aspect of the application is a non-transitory computer readable medium providing instructions for repeating sterilization cycles of a sterilization system as described herein, the instructions comprising: sensing a presence of a pathogen in a cell processing device; communicating the presence of the pathogen to a computer database; identifying the pathogen sensed in the cell processing device using the computer database; selecting a program of sterilization cycles from the computer database based on the identity of the pathogen; communication the selected program to a sterilization system described herein, wherein the sterilization apparatus is networked to automatically follow the program; performing the sterilization cycles according to the program, wherein each sterilization cycle comprises the steps of: providing a reservoir of a cleaning fluid; cavitating the reservoir of cleaning fluid by applying force to the cleaning fluid; generating a mist comprising aerosol droplets, wherein the mist is generated from the cleaning fluid while the cleaning fluid is subject to cavitation by force; subjecting the mist to a nonthermal plasma actuator to form plasma activated ionic particles; and contacting the plasma activated ionic particles to the airborne pathogen or pathogens on surfaces.

A further aspect of the application is a system described herein with an arc converter that uses pulses at a defined speed to provide better ionization.

Incorporated herein by reference is U.S. Pat. No. 11,376,587, which describes various devices, systems and methods compatible with the sterilization methods disclosed herein.

The following examples are by way of illustration only and should not be considered limiting on the aspects or embodiments of the application.

### Example 1

In a first test series, identical cultures of Serratia marcenscens were prepared by plating onto filter papers. One specimen was incubated for 24 hours at 30° C. in air as a control. Significant growth of the bacteria culture was observed. A second specimen was exposed to a 3 percent by volume aqueous hydrogen peroxide mist (which had not been activated) for 60 seconds in air at one atmosphere pressure, and thereafter incubated for 24 hours at 30° C. in air. Significant growth of the bacteria culture was observed. A third specimen was exposed to a 3 percent by volume aqueous hydrogen peroxide mist, which had been activated by passage through a 10.5 kilovolt AC arc, for 60 seconds in air at one atmosphere pressure, and thereafter incubated for 24 hours at 30° C. in air at one atmosphere pressure. This specimen showed no growth of the bacteria culture, which was killed by the treatment. After this demonstration that the activation treatment rendered the 3 percent hydrogen peroxide mist capable of preventing growth, additional respective specimens were tested using 1.5 percent, 0.75 percent, 0.3 percent, and 0 percent (“activated” water vapor only) concentration hydrogen peroxide mists for 60 seconds exposure in air at one atmosphere pressure, and incubated as described. The specimens contacted by the 1.5 percent and 0.75 percent hydrogen peroxide mists showed no growth. The specimen contacted by the 0.3 percent hydrogen peroxide mist showed very slight growth. The specimen contacted by the 0 percent hydrogen peroxide mist showed significant growth of the bacteria culture.

### Example 2

In an efficacy test, the sterilization device/system of the present disclosure was tested against a variety of bacterial spores and gram-negative bacteria (including multiple drug resistant organisms, gram-positive bacteria, mold and viruses. Using procedures described in the present disclosure, the log 10 reduction of the organisms in the following table were determined:

| | | Log |
|---|---|---|
| Organism | Classification | Reduction |
| Bacillus atrophaeus (surrogate for | Bacterial spore | >8.3 |
| B. anthracic) | | |
| Geobacillus stearotherophilus | Bacterial spore | >6.3 |
| Bacillus subtilis | Bacterial spore | >6.0 |
| Clostridium difficile | Bacterial spore | >6.0 |
| Escherichia coli | Gram Negative | >7.4 |
| Pseudomonas aeruginosa | Gram Negative | >6.0 |
| Serratia marcescens | Gram Negative | >6.0 |
| Salmonella entercia | Gram Negative | >5.5 |
| Staphylococcus aureus | Gram Positive | >7.4 |
| Methicillin- resistant Staphylococcus aureus | Gram Positive | >5.9 |
| Bacillus atrophaeus vegetative cells | Gram Positive | >9.0 |
| Aspergillus niger | Mold | >8.0 |
| Aspergillus species | Mold | >7.0 |
| Cladosporium species | Mold | >7.0 |
| Penicillium species | Mold | >7.0 |
| Stachybotrys chartarum | Mold | >7.0 |
| Trichophyton mentagrophytes | Mold | >6.0 |
| Human rhinovirus 16 (surrogate for human | Virus | >6.8 |
| influenza) | | |
| Influenza A (H1N1) | Virus | >10 |
| Norovirus | Virus | >6.4 |
| Adenovirus | Virus | >5.8 |

The results presented in the table show that the sterilization device/system of the present disclosure is an effective broad-spectrum surface and air disinfectant/decontaminant. It is effective against, bacterial spores, gram-negative bacteria, gram-positive bacteria, multiple drug resistant organisms, mold and viruses. The sterilization device/system is effective for mold mitigation and remediation, as well as the elimination of bacteria and viruses.

The sterilization cycle discussed herein relates to the conversion of hydrogen peroxide solution to ionized hydrogen peroxide after passing through an atmospheric cold plasma arc. Ionized hydrogen peroxide contains a high concentration of reactive oxygen species composed mostly of hydroxyl radicals. Reactive oxygen species damage pathogenic organisms through oxidation of proteins, carbohydrates, and lipids. This leads to cellular disruptions and/or dysfunction and allows for disinfection/sterilization in targeted areas, including large spaces.

In certain embodiments for direct application onto surfaces, the particle size for the ionized hydrogen peroxide is 0.5-3 microns, preferably on average 0.7 microns in diameter, flow rate is 50 ml per minute, dose application is 1 ml per square foot, with an application time of 5 seconds over per square foot of treatment area, and a contact time of 7 minutes to disinfect/decontaminate high touch surfaces.

In particular embodiments, the solution used is formulated as silver, chlorine and peracetic acid free, which maximizes material compatibility on rubber, metals, and other surfaces. Treatment time, dosage, dwell time, etc, can be varied to suit the desired sterilization goals of the user.

### Example 3

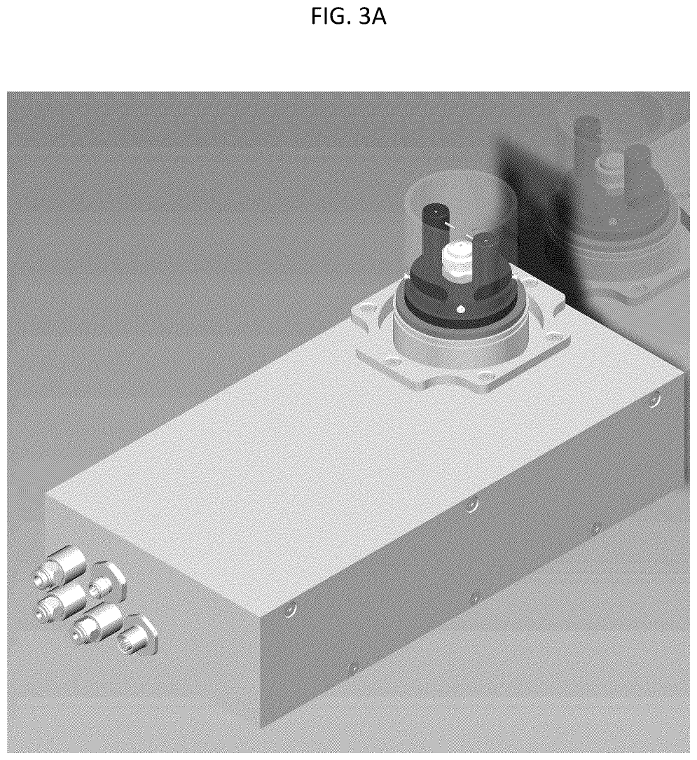

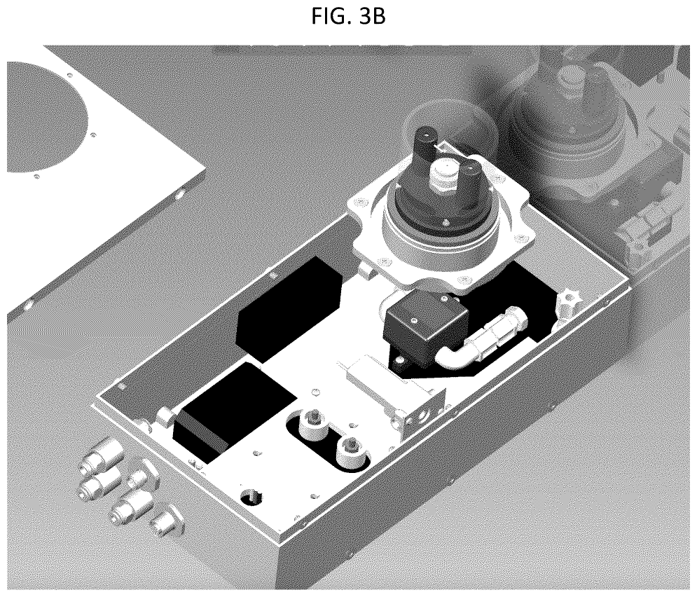

A custom closed space was created for a 90-degree applicator (see FIG. 3). The enclosure included the Keyence Flow Meter. The Keyence flow meter has greater range. The usual McMillian Flow Meter can only reduce to 13 ml/min. The Keyence can range as low as 2 ml/min.

When injecting into smaller spaces, utilizing low flow rates are required. While using the earlier nozzle bodies, an intermediate pulsation at low flow rates was observed because of a cavity within the nozzle and the nozzle body. To fix the problem it was necessary to eliminate the cavity, so there was a direct flow into the nozzle. Eliminating all cavities apart from that through which the very dry mist flows yielded a consistent flow as low as 2 ml/min.

A Deiner pump was used to achieve these lower flow rates within the system. Doing so also allowed lowering of the air pressure so that the pressure within the closed space was kept neutral. The Diener pump also provided a greater range in reducing as low as 2 ml/mi, vs the KNF pump, which is for higher ranges.

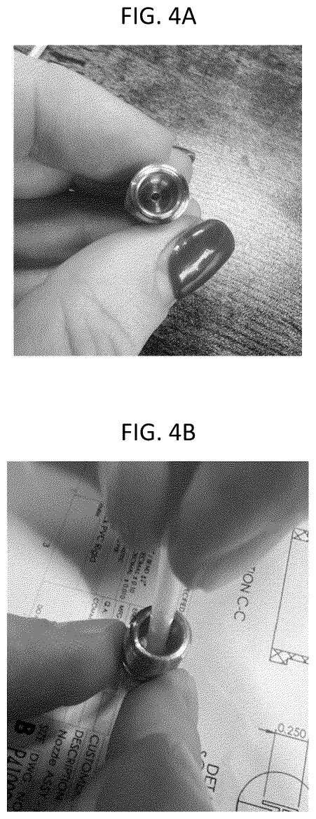

The earlier nozzle body was using ¼″ tubing/fitting, the self-cleansing nozzle body fitting is for ⅛″. The smaller size allowed for a steady stream of fluid at low flow rates. This modification helps with low-flow applications to remove a cavity that improves the spray consistency when reducing the flows at 2-3 ml/min. The previous tubing size was meant for higher flows at an average of 25 ml/min. In addition, another change to the nozzle body that was done was potting the fitting to reduce the internal diameter size of the fitting (see FIG. 4A). The modification was potting this this fitting with the tube with epoxy (see FIG. 4B).

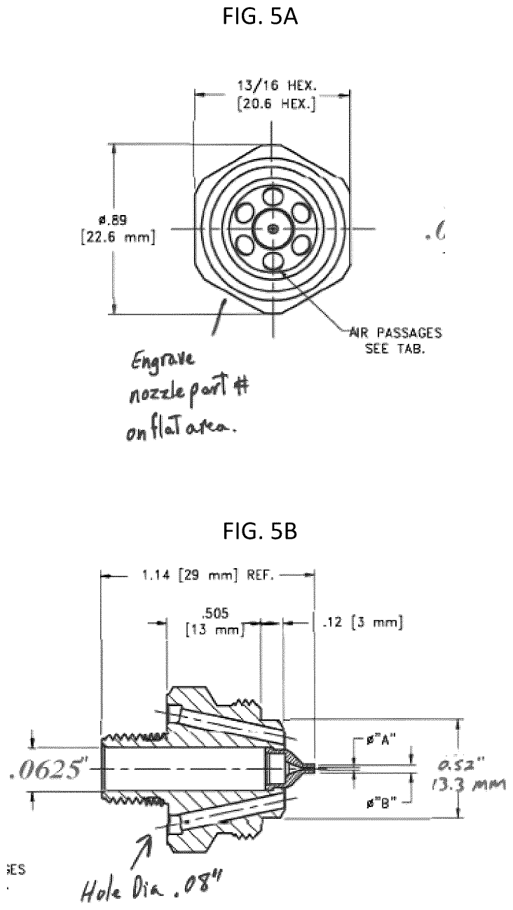

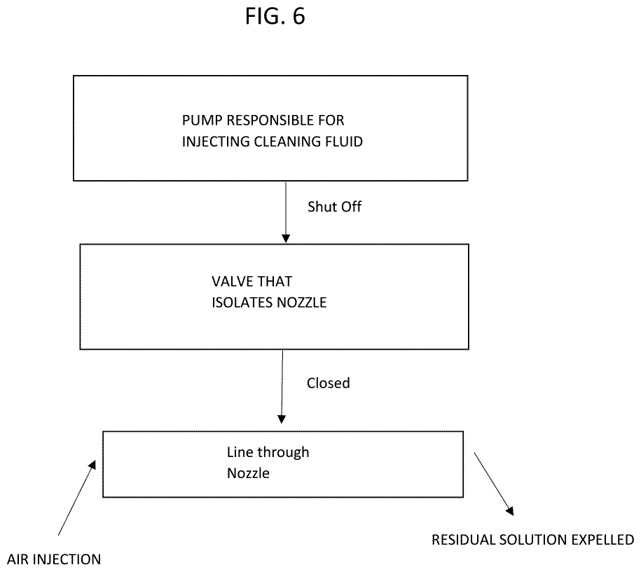

To further increase the efficacy, a smaller droplet size was implemented by using a 1050 psi or 850 psi nozzle instead of 1450 psi. Previously the 1050 psi or 850 psi could not be used with this application because of clogging issues. A self-cleaning nozzle was implemented to address this. The self-cleaning nozzle thread hole was changed to 0.0625″ (see FIGS. 5A and 5B). At the end of an injection cycle, the pump was shut off, the valve that isolates the nozzle was closed, and then air was injected into that line to blow out any residual solution (see FIG. 6). This makes the nozzle self-cleaning and eliminated any clogs within a nozzle as small as 1050 psi or even an 850 psi nozzle could obtain.

Although a particular embodiment of the invention has been described in detail for purposes of illustration, various modifications and enhancements may be made without departing from the spirit and scope of the invention. Accordingly, the invention is not to be limited except as by the appended claims.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A method for decontaminating a closed space, comprising the steps of:
entering input parameters of the closed space into a processing unit, wherein the processing unit is programmed to determine fluid properties of a cleaning fluid in a decontamination device based on the input parameters of the closed space;
activating a decontamination cycle of the decontamination device, wherein the decontamination cycle comprises the steps of: providing a reservoir of the cleaning fluid; setting the determined fluid properties of the cleaning fluid;
generating a very dry mist comprising cleaning fluid at substantially one atmosphere ambient pressure comprising aerosol droplets, wherein the cleaning fluid comprises a source of an active species for sterilization, wherein the active species is hydroxyl ions and wherein the source is hydrogen peroxide, and the cleaning fluid is silver-free chlorine-free and peracetic-acid free;
wherein the mist is subjected to a nonthermal plasma actuator to form plasma activated ionic particles carried by the aerosol droplets of the mist, wherein the plasma activated ionic particles are hydroxyl ions;
wherein a flow meter monitors a flow of the mist through a self-cleaning nozzle into the closed space, and wherein the flow rate of the mist through the self-cleaning nozzle is not greater than 2 ml/min;
wherein a pump controls flow rates and pressure through the self-cleaning nozzle by a pump, wherein air pressure within the closed space is maintained as neutral;
wherein the self-cleaning nozzle receives the flow of the mist through tubing of not greater than ⅛″ and there is no cavity permitted within the self-cleaning nozzle, wherein the generated very dry mist is applied to decontaminate the closed space.

2. The method of claim 1, further comprising operating the decontamination device manually.

3. The method of claim 2, wherein the decontamination device is hand-held to be operated manually.

4. The method of claim 1, wherein the input parameters of the closed space comprise at least one of: dimensions of the closed space, a position of the decontamination device relative to boundaries of the closed space, air temperature, pressure, and humidity of the closed space.

5. The method of claim 1, wherein the set fluid properties of the cleaning fluid comprise air pressure and fluid flow rate.

6. The method of claim 1, wherein the setting of the determined fluid properties to the cleaning fluid is performed by controlling an air valve.

7. The method of claim 6, wherein the air valve is controlled by programming the processing unit to control a potentiometer.

8. The method of claim 1, wherein the determined fluid properties of the cleaning fluid are adjusted by a size and a shape of a tube located at an exit of the cleaning fluid out of the decontamination device.

9. The method of claim 1, wherein the very dry mist comprises particles of diameter size in the range of 0.5-3 microns.

10. The method of claim 5, wherein the fluid properties of the cleaning fluid are set by lowering the air pressure and the fluid flow rate respectively below a predetermined standard air pressure and a predetermined standard fluid flow rate.

11. The method of claim 1, further comprising:
entering input parameters of a closed space into a processing unit, wherein the processing unit is further programmed to determine the fluid properties of the cleaning fluid in the decontamination device based on the input parameters of the closed space.

12. The method of claim 11, wherein the very dry mist comprises particles of diameter size in the range of 0.5-3 microns.

13. The method of claim 1, wherein the input parameters of the closed space are manually input.

14. The method of claim 1, wherein the input parameters of the closed space are measured by a plurality of sensors that are in networked communication with the processing unit.

15. The method of claim 1, wherein the processing unit and the decontamination device are in wireless communication.

16. A system for decontaminating a closed space, comprising a decontamination device and a computer processor, wherein the computer processor is in networked communication with the decontamination device,
wherein input parameters of the closed space are entered into the computer processor,
wherein the computer processor is programmed to determine fluid properties of a cleaning fluid in the decontamination device based on the input parameters of the closed space,
wherein the computer processor is further programmed to activate a decontamination cycle of the decontamination device, the decontamination cycle comprising the steps of:
providing a reservoir of the cleaning fluid;
setting the determined fluid properties of the cleaning fluid;
generating a very dry mist comprising cleaning fluid at substantially one atmosphere ambient pressure comprising aerosol droplets, wherein the cleaning fluid comprises a source of an active species for sterilization, wherein the active species is hydroxyl ions and wherein the source is hydrogen peroxide, and the cleaning fluid is silver-free chlorine-free and peracetic-acid free;
wherein the mist is subjected to a nonthermal plasma actuator to form plasma activated ionic particles carried by the aerosol droplets of the mist, wherein the plasma activated ionic particles are hydroxyl ions;
wherein a flow meter monitors a flow of the mist through a self-cleaning nozzle into the closed space, and wherein the flow rate of the mist through the self-cleaning nozzle is not greater than 2 ml/min;
wherein a pump controls flow rates and pressure through the self-cleaning nozzle by a pump, wherein air pressure within the closed space is maintained as neutral;
wherein the self-cleaning nozzle receives the flow of the mist through tubing of not greater than ⅛″ and there is no cavity permitted within the self-cleaning nozzle, wherein the generated very dry mist is applied to decontaminate the closed space.

17. The system of claim 16, wherein the decontamination device is operated manually.

18. The system of claim 17, wherein the decontamination device is hand-held to be operated manually.

19. The system of claim 16, wherein the input parameters of the closed space comprise: dimensions of the closed space, a position of the decontamination device relative to boundaries of the closed space, air temperature, pressure, and humidity of the closed space.

20. The system of claim 16, wherein the set fluid properties of the cleaning fluid comprise air pressure and fluid flow rate.
